(19) United States
(12) Patent Application Publication
(10) Pub. No.: US 2011/0112515 A1
(43) Pub. Date: May 12, 2011

(54) ACTUATED SELF UNPLUGGING SURGICAL SUCKER WAND

(76) Inventors: James B. Stiehl, Salem, IL (US); Kurt R. Stiehl, San Jose, CA (US)

(21) Appl. No.: 12/939,496

(22) Filed: Nov. 4, 2010

Related U.S. Application Data
(60) Provisional application No. 61/259,111, filed on Nov. 6, 2009.

(51) Int. Cl. A61M 1/00

(52) U.S. Cl. 604/540

(57) ABSTRACT

A suction wand for use during a surgical operation involving cutting of bone which yields bone debris, the wand comprising a suction conduit, a suction tip, a filter screen for retaining bone debris, and a carbon dioxide gas conduit for directing gas toward the suction tip to expel debris from the filter screen.

### CROSS-REFERENCE TO RELATED APPLICATIONS

This application is a non-provisional application of U.S. Ser. No. 61/259,111, filed Nov. 6, 2009, the entirety of which is incorporated herein by reference.

### FIELD OF THE INVENTION

The present invention generally relates to a suction device for use in surgical procedures and more particularly to a surgical suction device for removing debris from a surgery site.

### BACKGROUND OF THE INVENTION

Suction devices are commonly used in a variety of medical applications to evacuate various fluids from a patient for a variety of purposes. For example, during surgery, blood and other body fluids are commonly removed to permit visual inspection of the surgical site and disposal of fluids. Suction wands may also provide for the production of positive pressure (usually air or water) to irrigate the surgical site. One such example is disclosed in Lester, U.S. Pat. No. 4,526,573 which shows a suction conduit from sucking debris away from the surgery site and an irrigation conduit for directing irrigation fluid to a surgery site.

Prusmack US Publication No. 2007/0213667 discloses a suction irrigation cleaner configured to apply negative air pressure by connecting a first port to a suction source for removing debris and fluid from a surgical site. There is also an irrigation/positive pressure line to deliver positive air/vapor pressure by connecting a second port to a positive pressure source. The positive pressure fluid is used for irrigation and alternatively to unclog the device if tissue or clot is sucked into the device. The apparatus disclosed in Prusmack is not ideal because the tissue and clot can travel through device all the way to the valve mechanisms, potentially damaging the device. The irrigation function is complicated as it requires both turning of a stopcock and operation of a button to move the irrigation channel into the main flow path. Also, it does not inhibit larger potentially clogging debris from being sucked into the device beyond the point where the declogging function can be effective.

Historically, suction wands (commonly referred to as “sucker tips”) were fabricated from metal and were reusable only after suitable cleaning and sterilization. With the recent advent of disposable surgical equipment, however, metal suction wands have been almost entirely replaced by cheaper, light weight, disposable suction wand systems fabricated from plastics.

In orthopedic surgery, suction wands are vital for keeping the surgical site ‘dry’ improving visualization of tissues and for recovery of lost blood through means of ‘cell savers’ or devices that allow for the re-infusion of blood that may have oozed from the tissues of the surgical site. As shown in the Prusmack publication, a major obstacle to the optimal performance of sucker wands is the obstruction that occurs when bone bits and other debris are sucked into the suction line, effectively clogging the egress of fluids away from the surgical site and blocking the suction. A secondary problem has been the relatively high flow of air through the wand during the surgical procedure which leads to the potential deposition of airborne bacteria on the tip of the wand. And because the wand tip is moistened by the egress of fluid during suction, the wand tends to attract dust or debris which are then expelled to the surgical site when positive pressure is produced. Studies have shown that a sucker wand may be one of the highest sources of contamination during a surgical procedure.

Therefore, there exists a need for a suction wand device that allows for the egress of fluids being removed from a surgical site, but still provides the operator the ability to conveniently unclog or unplug the device without the fear of contaminating the surgical site or damaging the device. The present invention also offers an efficient design that can be constructed with disposable plastic materials and easily disassembled for cleaning purposes.

### SUMMARY OF THE INVENTION

Briefly, therefore, the invention is directed to a suction wand comprising a handle, a suction conduit at least partially disposed in the handle and partially defining a suction path, and a suction tip at an end of the suction conduit. The suction path extends from the suction tip and through the handle, wherein the suction conduit is adapted to communicate with a vacuum source at a vacuum source end of the suction path remote from the suction tip. There is a filter screen for retaining bone debris, wherein the filter screen is disposed in the suction path and has a first side facing a tip direction and an opposite second side facing a vacuum source direction. There is a CO₂ gas conduit adapted for fluid communication with a gas source and at least partially disposed in the handle and partially defining a CO₂ path, the CO₂ gas conduit including a CO₂ gas main conduit section through which CO₂ gas flows and is directed into the suction conduit in a direction toward the tip such that the CO₂ gas passes through the filter screen from the second side facing in the vacuum direction, in the tip direction and out the first side of the filter screen facing in the tip direction, for expelling debris from the first side of the filter. There is also a CO₂ gas conduit valve operable between an open position in which CO₂ gas flows through the CO₂ gas conduit and into the suction conduit toward the suction tip and a closed position in which CO₂ gas does not flow into the suction conduit.

In another aspect, the invention is directed to a suction wand for use in aspirating a surgical site during an orthopedic surgical operation which yields bone debris and generally comprises a handle and a suction conduit at least partially disposed in the handle and partially defining a suction path. A suction tip is at an end of the suction conduit. The suction path is adapted to communicate with a vacuum source at a vacuum source end of the suction path remote from the suction tip such that the suction path extends from the suction tip through the suction conduit to the vacuum source. A CO₂ gas conduit is adapted for fluid communication with a gas source and is at least partially disposed in the handle and partially defines a CO₂ path. A CO₂ gas conduit valve is operable between an open position in which CO₂ gas flows from the gas source through the CO₂ conduit and out the suction tip and a closed position in which CO₂ gas does not flow out of the suction tip. A filter screen is disposed in the suction path for retaining bone debris produced during the operation. The filter screen comprises a wire mesh including a set of parallel horizontal wires and a set of parallel vertical wires woven with the horizontal wires forming gaps between the wires. The gaps are sized and arranged to retain the bone debris on the screen when the wand is in a suction mode and permit a high blast of CO₂ to impinge upon the retained debris when the wand is in a CO₂ pressure mode to expel the debris from the screen.

Other objects and features will be in part apparent and in part pointed out hereinafter.

### BRIEF DESCRIPTION OF THE DRAWINGS

FIG. 1 is a perspective of a suction wand of the present invention;

FIG. 2A is a cross-section of the suction wand taken through line 2A-2A in FIG. 1 with a handle removed and showing a valve of the wand in a closed position;

FIG. 2B is a cross section of the suction wand taken through line 2B-2B in FIG. 1 with the handle removed and showing the valve in an open position;

FIG. 3 is an enlarged fragmentary cross section showing a suction conduit and a positive pressure conduit of the wand;

FIG. 4 is an enlarged front view of a filter screen and cap of the suction wand;

FIG. 5 is a schematic of a second embodiment of a suction wand;

FIG. 6 is a schematic of a third embodiment of a suction wand; and

FIG. 7 is a schematic of a fourth embodiment of a suction wand.

Corresponding reference characters indicate corresponding parts throughout the drawings.

### DESCRIPTION OF THE PREFERRED EMBODIMENTS

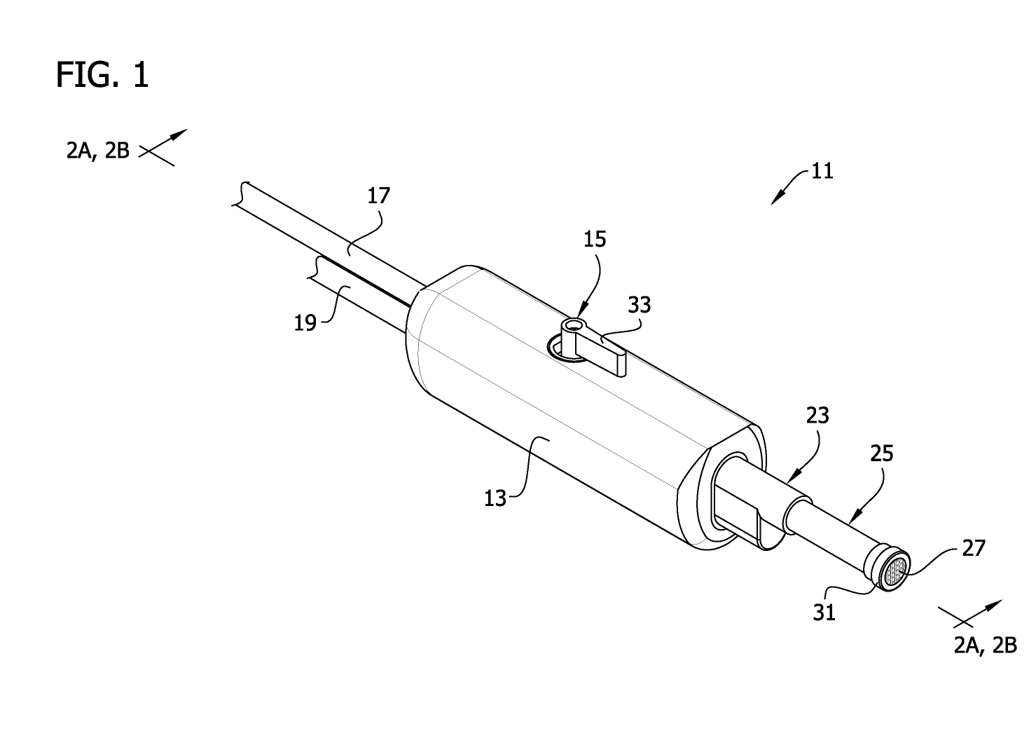

Referring to the illustrated embodiment, and in particular FIG. 1, a suction wand for use during surgical operation to aspirate a surgical site is generally indicated at 11. The suction wand comprises a handle 13 and a valve generally indicated at 15, partially received in the handle for movement between an open and a closed position. An inflow tube 17 extends proximally from a proximal end of the handle 13 and is configured for attachment to a positive pressure source (not shown). The preferred positive pressure source is a CO₂ tank. An outflow tube 19, below the inflow tube 17, also extends proximally from the proximal end of the handle 13 and is configured for attachment to a negative pressure or suction source (not shown).

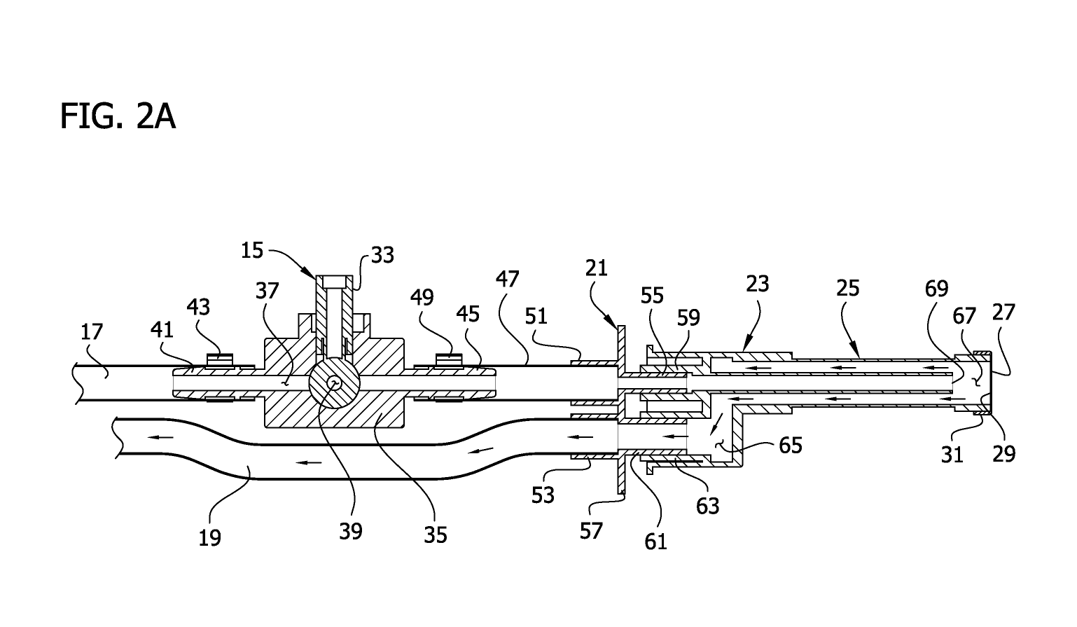

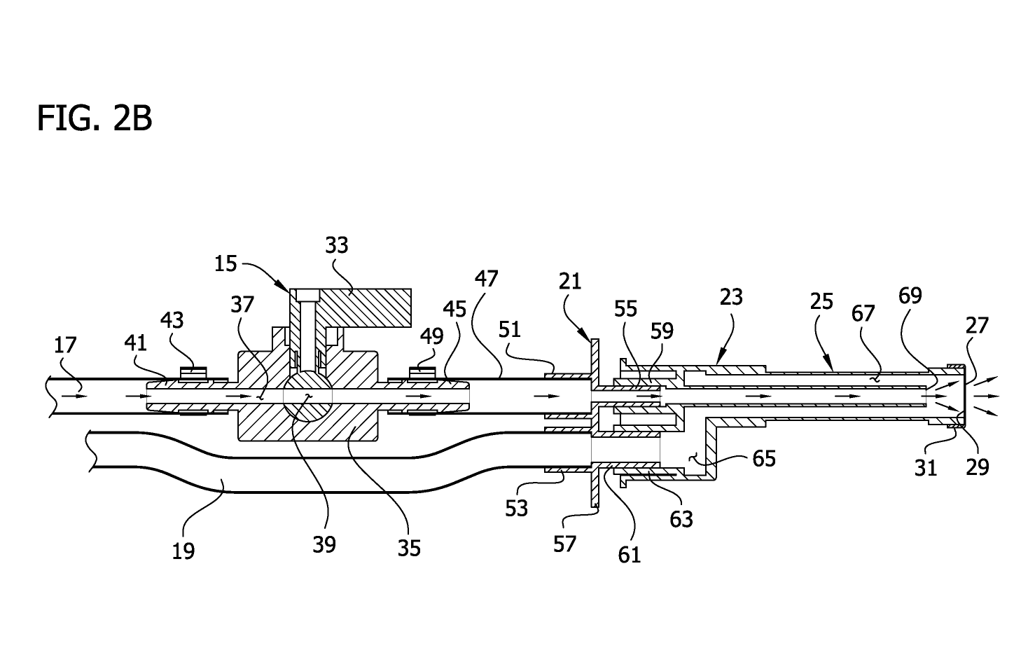

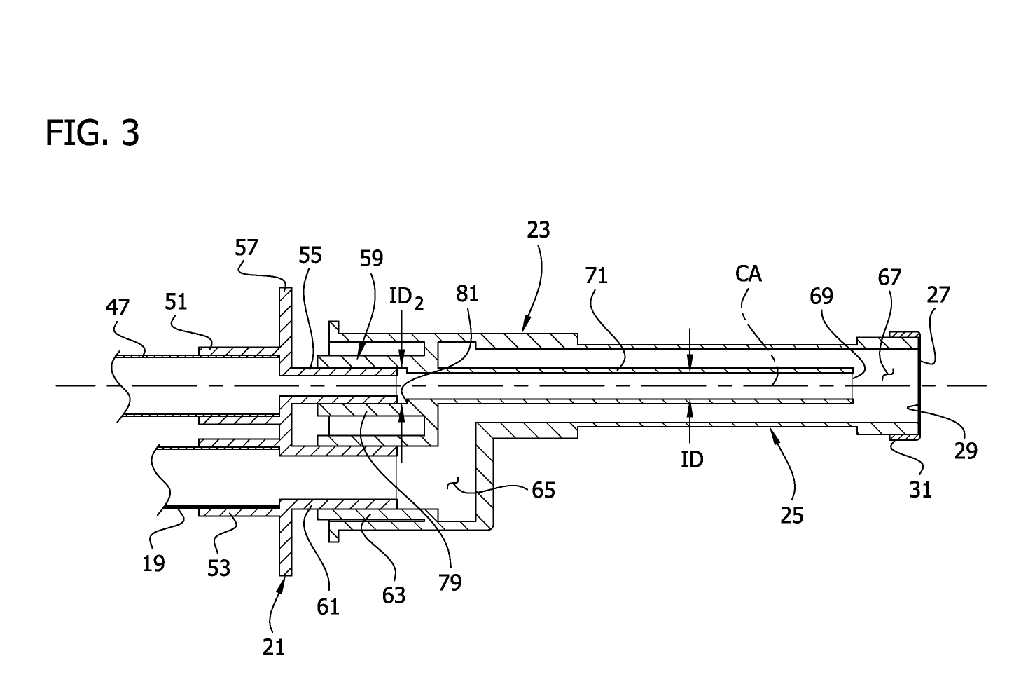

A mount 21 (FIGS. 2A-3) at a distal end of the handle 13 attaches a connector 23 and head portion 25 to the handle. The head portion 25 comprises a tubular member having a filter screen 27 disposed in an open, distal end or tip 29 of the head portion. A cap 31 retains the filter screen 27 in the tip 29. The cap 31 can be removable to allow for the replacement of the filter screen 27 after use. As will be explained in greater detail below, when the valve 15 is in the closed position (FIG. 2A), activation of the suction source will generate a negative pressure gradient at the tip 29 of the head portion 25 for aspirating the surgical site when the wand 11 is used in a surgical procedure. Movement of an actuator 33 of the valve 15 moves the valve from the closed position to the open position (FIG. 2B), permitting fluid from the positive pressure source to escape through the tip 29 of the head portion 25, past the filter screen 27 to expel any debris collected on the screen during aspiration. In the illustrated embodiment, the valve 15 is a ball valve. The handle 13, mount 21, connector 23, head portion 25 and cap 31 are formed from plastic. However, the components can be made from other suitable materials such as stainless steel.

Referring to FIGS. 2A and 2B, a valve housing 35 is disposed in the handle 13 and seats the valve 15 for pivotal movement in the valve housing between the open and closed positions. The valve housing 35 includes a valve passage 37 that extends longitudinally in the valve housing and is partially defined by an opening 39 in the valve 15. An inlet port 41 extends proximally from a proximal end of the valve housing 35 and is received in a distal end of the inflow tube 17 to fluidly connect the inflow tube to the valve passage 37. A clamp 43 secures the inflow tube 17 around the inlet port 41. An outlet port 45 extends distally from a distal end of the valve housing 35 and is received in a proximal end of a connection tube 47 to fluidly connect the connection tube to the valve passage 37. A clamp 49 secures the connection tube 47 around the outlet port 45. A distal end of the connection tube 47 is received in a first connection port 51 of the mount 21 to fluidly connect the connection tube to the mount. A second connection port 53 of the mount 21, below the first connection port 51, receives a distal end of the outflow tube 19 to fluidly connect the outflow tube to the mount. In the illustrated embodiment, the connection tube 47 and outflow tube 19 are secured in the connection ports 51, 53, respectively, by adhesive. However, other suitable connection means such as clamps or by friction fit are within the scope of the present invention. Also, connectors other than clamps 43, 49 can be used.

A first mount port 55 extends distally from a base 57 of the mount 21 and is received in a first connector port 59 of the connector 23 to fluidly connect the first mount port to the connector. A second mount port 61 of the mount 21, below the first mount port 55, also extends distally from the base 57 and is receive in a second connector port 63 of the connector 23 to fluidly connect the second mount port to the connector.

The first connector port 59 extends distally through the connector 23 into the head portion 25. The first mount port 55 and first connector port 59 broadly define a positive pressure conduit. The second connector port 63 opens into an interior space 65 in the connector 23 which opens into an interior space 67 in the head portion 25. The second mount port 61, second connector port 63, connector interior space 65 and head portion interior space 67 broadly define a suction conduit. The suction conduit extends around the positive pressure conduit to the tip 29 of the head portion 25. In this embodiment the longitudinal axes of the suction conduit and positive pressure conduit are coincident such that they are disposed on a common central axis CA (FIG. 3). In the illustrated embodiment, an open end or tip 69 of the positive pressure conduit is spaced between about ¼ and about ¾ in., e.g., about ½ in., from the filter screen 27, the reasons for which will be explained in greater detail below. It is seen there that in once sense the central axis of the positive pressure conduit, which here is the CO₂ gas conduit, and in particular the segment of the conduit which is the CO₂ gas conduit extension, is parallel with the central axis of the suction conduit, at least in the segment from the suction tip back to the handle. In another aspect it is seen that the central axis of the positive pressure conduit, which here is the CO₂ gas conduit, and in particular segment of the conduit which is the CO₂ gas conduit extension, is coincident with the central axis of the suction conduit, at least in the segment from the suction tip back to the handle.

Referring to FIG. 2A, a suction path is illustrated by a series of arrows. The suction path extends from a distal side of the filter screen 27 past the screen and tip 29 of the head portion 25, through the suction conduit and into the outflow tube 19 to the suction source. When the suction wand 11 is used during a surgical procedure to aspirate a surgical site, the tip 29 of the head portion 25 is placed adjacent the surgical site and fluid and small particles permitted by the filter screen 27 are sucked from the surgical site along the suction path and can be collected remotely from the suction wand. To prevent fluid and debris from entering the positive pressure conduit a grate (not shown) can optionally be placed over the tip 69 of the positive pressure conduit.

Conversely, a positive pressure path is illustrated by a series of arrows in FIG. 2B. The positive pressure path extends from the positive pressure source, through the inflow tube 17, into the inlet port 41 of the valve housing 35, through the valve passage 37, out the outlet port 45, into the connection tube 57, past the first connection port 51 of the mount 21, through the first mount port 55, into the first connector port 59 of the connector 23, through the positive pressure conduit, out the tip 69 of the positive pressure conduit, partially through the interior space 67 of the head portion 25 and out the tip 29 of the head portion, past the filter screen 27. The conduit segment between port 55 and tip 69 constitutes a CO₂ gas conduit extension which extends within an interior of the suction conduit for directing CO₂ gas directly at the suction tip. The CO₂ gas conduit extension in the preferred embodiment shown has a smaller inner diameter than the inner diameter of the CO₂ gas main conduit section, as manifest by tubes 47 and 17. After the surgical site has been aspirated, the suction wand 11 can be operated in the positive pressure mode to expel any debris, such as larger bone chips in the case of orthopedic surgery, which may be caught in the filter screen 27. Referring to FIG. 3, a distal end 71 of the first connector port 59 has an inner diameter ID of about 3/32 in. The inner diameter ID of the distal end 71 of the first connector port 59 can be between about 1/16 and about ⅛ in., the purpose of which will be explained in greater detail below.

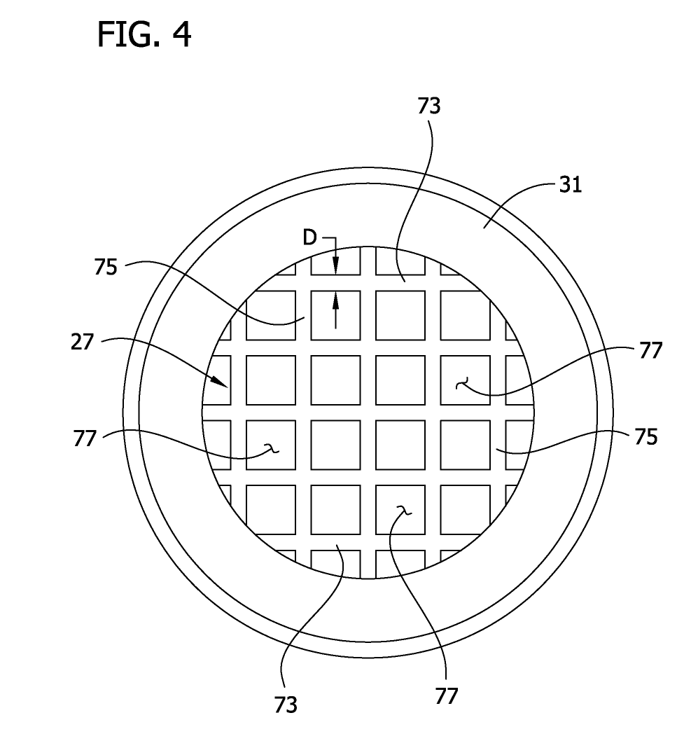

Referring to FIG. 4, the filter screen 27 comprises a woven mesh of parallel horizontal wires 73 and parallel vertical wires 75 forming open areas 77. The wires 73, 75 may have a diameter D between about 0.01 in. and about 0.02 in. The wires 73, 75 can be spaced such that there are between about 12 to about 30 open areas 77 per square in. In the illustrated embodiment, there are about 20 open areas 77. The open areas 77 can make up between about 49% and about 54% of the surface area of the filter screen 27. The wires 73, 75 of the filter screen 27 are arranged such that the open areas 77 are sized and spaced to prevent debris from entering the suction conduit during aspiration of a surgical site yet still provide a sufficient opening to deliver a maximum output flow when the wand 11 is in the positive pressure delivery mode. In a preferred embodiment the wires 73, 75 are metal (i.e., steel, copper, brass, platinum). However, the wires 73, 75 can also be made from plastic, nylon or any other suitable material.

In use, the inflow tube 75 is preferably connected to a positive pressure source comprising a CO₂ tank. Carbon dioxide is preferred because of its safety, surgical compatibility, and availability. Additionally, the relatively small inner diameter ID of the distal end 71 of the first connector port 59 (about 3/32 in.) produces a high gas flow at the tip 29 of the head portion 25. As shown in FIG. 3, a proximal end 79 of the first connector port 59 has an inner diameter ID₂ that is greater than the inner diameter ID of the distal end 71 of the first connector port forming a shoulder 81 in the first connector port. This constriction in conduit area, under the Venturi principle, with a CO₂ pressure typically at 50 psi dramatically increases the air flow at the tip 29 of the head portion 25. The space between the tip 69 of the positive pressure conduit and the tip 29 of the head portion 29 also controls the pressure at the tip of the head portion. The increase in pipe area that occurs in the transition between the tip 69 of the positive pressure conduit and the tip 29 of the head portion 25 will slightly decrease the pressure seen at the head portion tip. An additional mechanism to guarantee high air pressure at the tip 29 of the wand 11 is to clamp the outflow tube 19 with a hose clamp (not shown). This will create a closed pressure system.

Also, because the longitudinal axis of the positive pressure conduit is coincident with the longitudinal axis of the suction conduit, the CO₂ blast produced by the positive pressure source will get a “running start” since there are no turns or bends in the CO₂ path which could slow down the gas flow. The linear CO₂ path also reduces the risk of any “dead spots” that can result from a build up of turbulence at the bends. Finally, the alignment of the positive pressure conduit and suction conduit ensures that the blast of CO₂ impacts the filter screen 27 in a directly perpendicular manner.

The high pressure, focused blast of CO₂ that results from this construction is needed so that a sufficient amount of gas impinges upon any captured debris in the filter screen 27 to expel the debris. The open areas 77 in the filter screen 27 ensure that a sufficient amount of air can impinge upon the captured debris to expel the debris from the screen. It was found that the range of about 12 to about 30 open areas 77 per square in. is the preferred range to retain bone chips large enough to clog the wand 11 while still providing enough open area to produce a high blast of CO₂ through the screen 27 to expel the debris, and enough open area to provide the desired suction in normal operation.

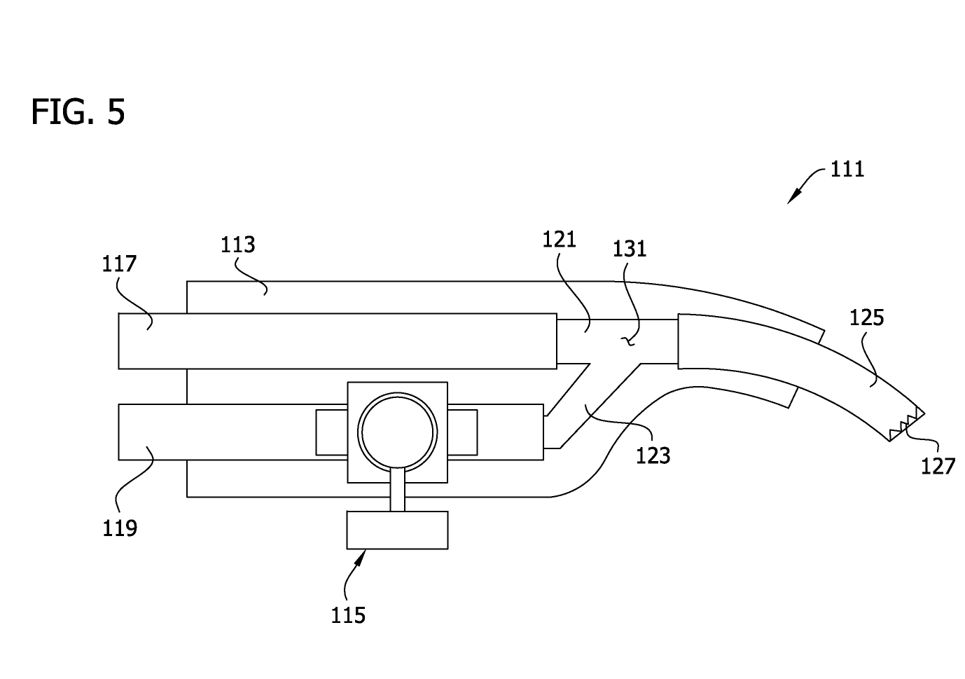

A second embodiment of a suction wand 111 of the present invention is shown in FIG. 5. The wand 111 of the second embodiment comprises a handle 113 and a valve 115 partially received in the handle moveable between an open and closed position. In the illustrated embodiment, the valve 115 is a ball valve. In the open position, an inflow tube 117 can deliver positive pressure from a positive pressure source (not shown) to a positive pressure conduit 121 in the handle 113. The positive pressure conduit 121 extends through the handle 113 out a distal end of the handle to a tip portion 125. A filter screen 127 is disposed in an open, distal end of the tip portion 125. The wand 111 is configured to deliver a positive pressure force at the tip portion 125 of about 50 psi.

An outflow tube 119 extends through the handle 113 and connects to a suction conduit 123 in the handle. The suction conduit 123 also extends through the handle 123 and out the distal end of the handle to the tip portion 125. The positive pressure conduit 121 and suction conduit 123 converge in the handle 113 at a permanent Y-junction 131. In the closed position, the outflow tube 119 can create a negative pressure gradient at the tip portion 125 by activation of a suction source (not shown) in fluid communication with the outflow tube.

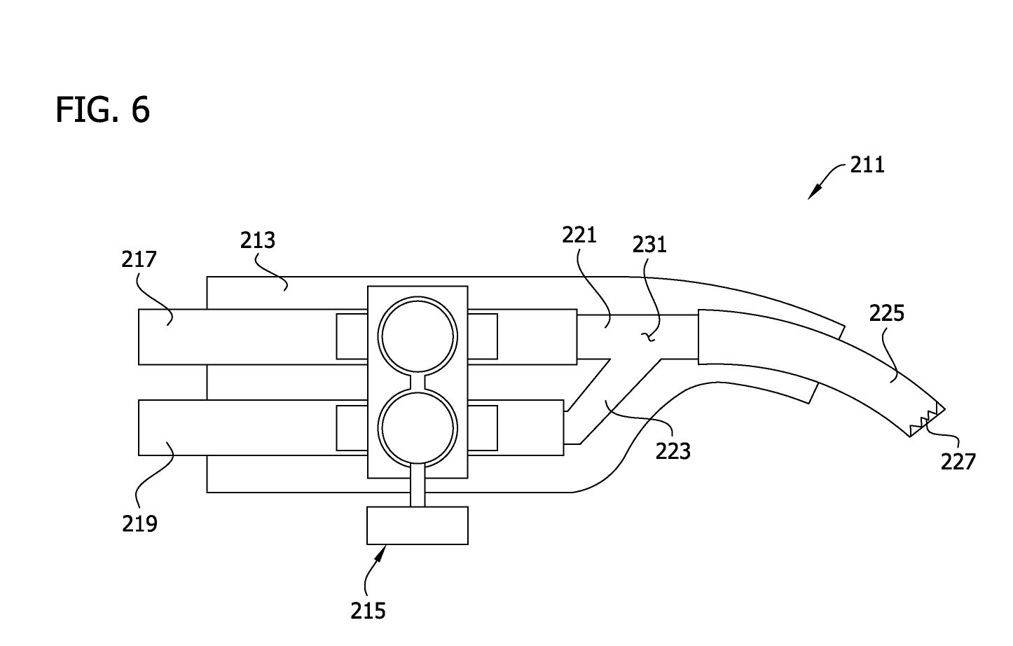

A third embodiment of a suction wand 211 of the present invention is shown in FIG. 6. The third embodiment is substantially the same as the second embodiment except for valve 215 which is configured to control the delivery of pressure in both the suction conduit 223 and the positive pressure conduit 221. The valve 215 is configured such that in a first position the valve closes the positive pressure conduit 221 and opens the suction conduit 223. In a second position, the valve 215 closes the suction conduit 223 and opens the positive pressure conduit 221. In the illustrated embodiment, the valve 215 is a double-ball valve. However, other suitable valves can be used within the scope of the invention.

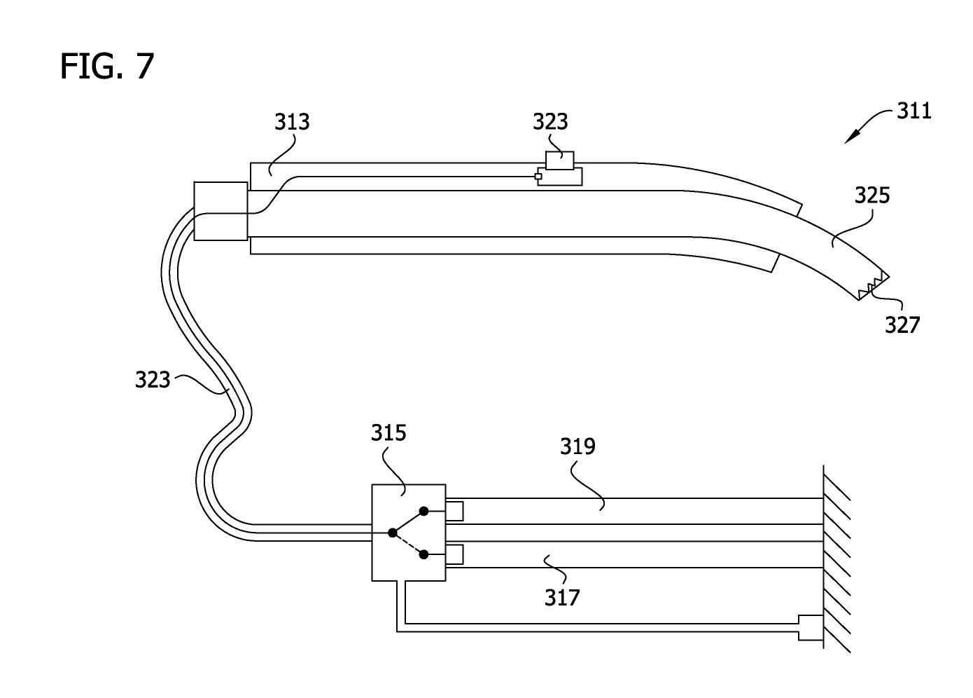

A fourth embodiment of a suction wand 311 of the present invention is shown in FIG. 7. The wand 311 of the fourth embodiment comprises an inflow tube 317 and an outflow tube 319 adapted for connection to respective positive pressure and negative pressure sources. The tubes 317, 319 are connected to a switch 315 at their distal ends. A hose 323 extends from the switch and connects to a positive pressure/suction conduit 321 received in a handle 313 of the wand 311. A button 232 on the handle 313 is depressable to move the switch 315 between the inflow and outflow tubes 317, 319 to selectively communicate one of the tubes with the hose 232. A tip portion 325 extends from a distal end of the handle 313 and has a filter screen 327 disposed in an open distal end. This embodiment is similarly configured to generate a positive pressure force of about 50 psi at the open distal end of the tip portion 325.

Having described the invention in detail, it will be apparent that modifications and variations are possible without departing from the scope of the invention defined in the appended claims.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions without departing from the scope of the invention, it is intended that all matter contained in the above description and shown in the accompanying drawings shall be interpreted as illustrative and not in a limiting sense.

What is claimed is:

1. A suction wand for use in aspirating a surgical site during a surgical operation involving cutting of bone which yields bone debris, the suction wand comprising:
a handle;
a suction conduit at least partially disposed in the handle and partially defining a suction path;
a suction tip at an end of the suction conduit, wherein the suction path extends from the suction tip and through the handle, wherein the suction conduit is adapted to communicate with a vacuum source at a vacuum source end of the suction path remote from the suction tip;
a filter screen for retaining bone debris, wherein the filter screen is disposed in the suction path and has a first side facing a tip direction and an opposite second side facing a vacuum source direction;
a CO₂ gas conduit adapted for fluid communication with a gas source and at least partially disposed in the handle and partially defining a CO₂ path, the CO₂ gas conduit including a CO₂ gas main conduit section through which CO₂ gas flows and is directed into the suction conduit in a direction toward the tip such that the CO₂ gas passes through the filter screen from the second side facing in the vacuum direction, in the tip direction and out the first side of the filter screen facing in the tip direction, for expelling debris from the first side of the filter; and
a CO₂ gas conduit valve operable between an open position in which CO₂ gas flows through the CO₂ gas conduit and into the suction conduit toward the suction tip and a closed position in which CO₂ gas does not flow into the suction conduit.

2. The suction wand of claim 1 comprising a CO₂ gas conduit extension which extends within an interior of the suction conduit for directing CO₂ gas directly at the suction tip.

3. The suction wand of claim 2 wherein the CO₂ gas conduit extension has a smaller inner diameter than an inner diameter of the CO₂ gas main conduit section.

4. The suction wand of claim 2 wherein a central axis of the suction conduit is coincident with a central axis of the CO₂ gas conduit extension.

5. The suction wand of claim 3 wherein the inner diameter of the CO₂ gas conduit extension is between about 1/16 in. and about ⅛ in.

6. The suction wand of claim 3 wherein the inner diameter of the CO₂ gas conduit extension is about 3/32 in.

7. The suction wand of claim 2 wherein the CO₂ gas conduit extension terminates at a tip spaced between about ¼ and about ¾ from the filter screen.

8. The suction wand of claim 1 wherein the filter screen is in the suction conduit.

9. The suction wand of claim 7 wherein the filter screen is at the suction tip.

10. The suction wand of claim 1 wherein the filter screen comprises a wire mesh including a set of parallel horizontal wires and a set of parallel vertical wires woven with the horizontal wires forming gaps between the wires, the gaps being sized and arranged to retain debris on the screen when the wand is in a suction mode and permit a high blast of CO₂ to impinge upon the retained debris when the wand is in a CO₂ pressure mode to expel the debris from the screen.

11. The suction wand of claim 8 wherein both the horizontal and vertical wires have a cross-sectional diameter between about 0.010 in. and about 0.02 in.

12. The suction wand of claim 8 wherein the gaps make up between about 49% and about 54% of a total surface area of the filter screen.

13. The suction wand of claim 2 wherein the CO₂ gas conduit extension has a central axis which is parallel with a central axis of the suction conduit, at least in a segment from the suction tip back to the handle.

14. The suction wand of claim 2 wherein the CO₂ gas conduit extension has a central axis which is coincident with a central axis of the suction conduit, at least in a segment from the suction tip back to the handle.

15. The suction wand of claim 1 wherein the CO₂ gas conduit valve is a ball valve.

16. The suction wand of claim 1 further comprising a suction conduit valve integral with the CO₂ gas conduit valve, wherein the suction conduit valve is in a closed position when the CO₂ gas conduit valve is in its open position, and wherein the suction conduit valve is in an open position when the CO₂ gas conduit valve is in its closed position.

17. The suction wand of claim 1 wherein when the CO₂ conduit is connected to the gas source the CO₂ conduit is configured to produce a gas pressure of about 50 psi at the suction tip.

18. The suction wand of claim 1 comprising a permanent Y-junction between the CO₂ gas conduit and the suction conduit permitting the directing of CO₂ gas into the suction conduit in a direction toward the tip for expelling debris from the first side of the filter.

19. A suction wand for use in aspirating a surgical site during an orthopedic surgical operation which yields bone debris, the suction wand comprising:
a handle;
a suction conduit at least partially disposed in the handle and partially defining a suction path;
a suction tip at an end of the suction conduit, wherein the suction path is adapted to communicate with a vacuum source at a vacuum source end of the suction path remote from the suction tip such that the suction path extends from the suction tip through the suction conduit to the vacuum source;
a CO₂ gas conduit adapted for fluid communication with a gas source and at least partially disposed in the handle and partially defining a CO₂ path;
a CO₂ gas conduit valve operable between an open position in which CO₂ gas flows from the gas source through the CO₂ conduit and out the suction tip and a closed position in which CO₂ gas does not flow out of the suction tip; and
a filter screen disposed in the suction path for retaining bone debris produced during the operation, wherein the filter screen comprises a wire mesh including a set of parallel horizontal wires and a set of parallel vertical wires woven with the horizontal wires forming gaps between the wires, the gaps being sized and arranged to retain the bone debris on the screen when the wand is in a suction mode and permit a high blast of CO₂ to impinge upon the retained debris when the wand is in a CO₂ pressure mode to expel the debris from the screen.

20. The suction wand of claim 16 wherein the filter screen is at the suction tip.

21. The suction wand of claim 16 wherein the gaps make up between about 49% and about 54% of a total surface area of the filter screen.

22. The suction wand of claim 16 wherein the filter screen comprises between about 12 and about 30 gaps per square in.

23. The suction wand of claim 16 wherein both the horizontal and vertical wires have a cross-sectional diameter between about 0.010 in. and about 0.02 in.

24. A suction wand for use in aspirating a surgical site during a surgical operation involving cutting of bone which yields bone debris, the suction wand comprising:
a handle;
a suction conduit at least partially disposed in the handle and partially defining a suction path;
a suction tip at an end of the suction conduit, wherein the suction path extends from the suction tip and through the handle, wherein the suction conduit is adapted to communicate with a vacuum source at a vacuum source end of the suction path remote from the suction tip;
a filter screen for retaining bone debris, wherein the filter screen is disposed in the suction path and has a first side facing a tip direction and an opposite second side facing a vacuum source direction;
a CO₂ gas conduit adapted for fluid communication with a gas source and at least partially disposed in the handle and partially defining a CO₂ path, the CO₂ gas conduit including a CO₂ gas main conduit section through which CO₂ gas flows and is directed into the suction conduit in a direction toward the tip such that the CO₂ gas passes through the filter screen from the second side facing in the vacuum direction, in the tip direction and out the first side of the filter screen facing in the tip direction, for expelling debris from the first side of the filter;
a CO₂ gas conduit extension which extends within an interior of the suction conduit for directing CO₂ gas directly at the suction tip, wherein a central axis of the suction conduit is coincident with a central axis of the CO₂ gas conduit extension, and the CO₂ gas conduit extension terminates at a tip spaced between about 1/4 and about 3/4 from the filter screen;
a CO₂ gas conduit valve operable between an open position in which CO₂ gas flows through the CO₂ gas conduit and into the suction conduit toward the suction tip and a closed position in which CO₂ gas does not flow into the suction conduit.
